SEQUENCE LISTING (12 sequences)

SEQ ID NO: 1 - protein, 16 aa, Saccharomyces cerevisiae
AVDIWSLGCIFAEMVT

SEQ ID NO: 2 - protein, 16 aa, Saccharomyces cerevisiae
GVDTWSIGCIFAEMCN

SEQ ID NO: 3 - protein, 16 aa, Saccharomyces cerevisiae
AVDWWALGVLIYEMAA

SEQ ID NO: 4 - protein, 16 aa, Saccharomyces cerevisiae
TIDAWALGVLAFELLT

SEQ ID NO: 5 - protein, 16 aa, Saccharomyces cerevisiae
EVDIWSLGVMLYALLI

SEQ ID NO: 6 - protein, 16 aa, Saccharomyces cerevisiae
LVDMWSMGCLVYVILT

SEQ ID NO: 7 - protein, 16 aa, Saccharomyces cerevisiae
LSDIWSLGATVVEMLT

SEQ ID NO: 8 - protein, 16 aa, Saccharomyces cerevisiae
KADIWSTGCVVIEMFT

SEQ ID NO: 9 - protein, 16 aa, Saccharomyces cerevisiae
KVDIWSLGIMIIEMIE

SEQ ID NO: 10 - protein, 16 aa, Saccharomyces cerevisiae
TVDYWSLGCMLFESLV

SEQ ID NO: 11 - protein, 16 aa, Saccharomyces cerevisiae
LSDIWSLGCVIFEMCS

SEQ ID NO: 12 - protein, 16 aa, Saccharomyces cerevisiae
KADIFSLGLMIVEIAA

(19) United States
(12) Patent Application Publication
(10) Pub. No.: US 2014/0065650 A1
(43) Pub. Date: Mar. 6, 2014

(54) METHOD FOR THE REGULATION OF PROTEIN KINASE ACTIVITY IN VIVO AND IN VITRO

(71) Applicants: Damien D'Amours, Montreal (CA); Hery Ratsima, Montreal (CA)

(72) Inventors: Damien D'Amours, Montreal (CA); Hery Ratsima, Montreal (CA)

(73) Assignee: UNIVERSITÉ DE MONTRÉAL, Montreal, QC (CA)

(21) Appl. No.: 14/116,425

(22) PCT Filed: May 9, 2012
(86) PCT No.: PCT/CA2012/000442

§ 371 Date: Nov. 8, 2013

Related U.S. Application Data
(60) Provisional application No. 61/483,839, filed on May 9, 2011.

(51) Int. Cl. C12Q 1/48

(52) U.S. Cl. CPC C12Q 1/485
USPC 435/15; 435/194; 536/23.2; 435/320.1; 435/254.2

(57) ABSTRACT

Disclosed herein is a substantially pure nucleic acid encoding a eukaryotic protein kinase having at least one mutated amino-acid residue located in its subdomain IX. Also disclosed is a substantially pure eukaryotic protein kinase polypeptide having at least one mutation in its subdomain IX, the kinase being encoded by the nucleic acid.

### TECHNICAL FIELD

The present relates to mutated eukaryotic protein kinases, and more particularly to domain specific mutated eukaryotic protein kinases and uses thereof.

### BACKGROUND

Reversible protein phosphorylation plays critical roles in the regulation of most physiological processes in eukaryotes (1). This modification is catalyzed by protein kinases, a large family of enzymes regulating nearly every aspect of cell biology. The main structural features of protein kinases are highly conserved among various members of this family of proteins, and yet individual kinases display very high specificity in their modes of activation and substrate selection in vivo (1). Precise spatiotemporal regulation of protein kinase activity has been achieved, at least in part, through association of kinase domains with ancillary targeting domains and/or regulatory subunits (2, 3). Understanding the molecular mechanisms goveming enzymatic activation and substrate specificity in multidomain kinases depends largely on the ability to dissociate the activity of regulatory and catalytic domains in these enzymes.

It is well established that multiple human diseases are connected to defects in kinases or protein phosphorylation. One of the best known examples of diseases presenting those defects is cancer. Specifically, most advanced cancers in humans are characterized by an aberrant number of chromosomes. This aberrant number of chromosomes, or aneuploidy, is caused by the misregulation of chromosome segregation during mitosis. Conserved cell cycle kinases play critical roles in the regulation of chromosome segregation in all animal cells. Interestingly, chemical inhibition of mitotic kinases is also a key therapeutic approach to kill cancer cells in humans. It is thus important to understand how mitotic kinases promote effective cell division in order to understand how their misregulation can promote tumorigenesis or can be used to treat cancers.

There are several protein kinase families known to be major regulators of cell division and chromosome segregation. Specifically, cyclin-dependent kinases (CDKs), polo-like kinases (PLKs), and the aurora kinase have all been directly implicated in the generation of aneuploidy and cancer progression when misregulated in human cells. Understanding how cancer development is promoted or suppressed as a result of kinase misregulation thus depends on our ability to functionally characterize the basic mechanisms of action of these families of kinases in human cells.

Polo-like kinases (PLKs) are multidomain kinases playing essential roles in cell division, proliferation and development (4). A defining feature of this family of kinases is the presence at the protein carboxy terminus of a phosphopeptide interaction module, the polo-box domain (PBD). The PBD is believed to stimulate phosphorylation of PLK substrates by mediating phospho-dependent interactions between PLKs and its substrates (5). The PBD can also target PLKs to specific subcellular domains, such as centrosomes (6, 7), where the increased local concentration of the kinase promotes the phosphorylation of specific substrates. Interestingly, a growing number of studies have shown that the PBD is capable of mediating interactions with unphosphorylated proteins as well (5). One such interaction involves the kinase domain of PLKs, and is believed to suppress the activity of the kinase until a substrate is bound to the PBD (8). This intramolecular interaction does not require residues important for phosphopeptide-binding, thereby suggesting the existence of distinct binding modes for the association of phosphorylated and unmodified proteins with the PBD (9). The PBD thus appears to be a key interaction hub for the regulation of PLK localization, substrate-specificity and enzymatic activity.

How the kinase domain and the PBD of PLKs work together to promote cell cycle progression in living cells is unclear. In particular, it is unknown whether the PBD plays kinase-independent functions in vivo (4). Discovering the specific roles of PLK functional domains in cells is a formidable challenge because kinase inactivation leads to lethality and thus prevents detailed in vivo analyses. Indeed, it has not been possible so far to isolate PLK mutants constitutively defective in kinase activity. Furthermore, existing conditional mutations have undesirable side effects, such as impaired proliferation or changes in protein levels. Cell cycle kinases are uniquely difficult to study because they are both essential for viability and act in a sequential manner during cell division. This means that complete inactivation of these kinases kills cells and thus prevents further characterization of their roles in vivo. In addition, the fact that kinases play multiple roles at different stages of the cell cycle means that inhibition of an early event in the cell cycle prevents analysis of a later event (e.g., inhibition of S phase by inactivation of CDKs prevents analysis of their roles in M phase).

In light of the above, there is an important need to develop novel strategies that will enable researchers to inactivate essential kinases in a conditional manner (to maintain viability of cells) with high temporal resolution (to allow analysis of both early and late events in the cell cycle). To address this goal, we have developed a completely novel approach to study kinase activity in vivo and in vitro using engineered mutations that allow one to control kinase catalytic activity at will. This new approach will allow scientists to probe the specific molecular contribution of several cell cycle kinases to cell division, chromosome segregation, and ultimately, to cancer development.

### BRIEF SUMMARY

We have developed a novel approach to quantitatively control protein kinase activity in vivo and in vitro. This novel approach, which we term “the kinant approach”, uses engineered mutations at specific positions in the conserved F-helix region (i.e., subdomain IX) of several cell cycle kinases to confer thermosensitivity on those kinases. This thermosensitivity can be used to modulate or completely abrogate the enzymatic activity of essential protein kinases at specific stages of the cell cycle. Importantly, we have demonstrated that the activity of purified kinases carrying kinant mutations can be quantitatively controlled with temperature in vitro. Consistent with this, cells carrying kinant versions of essential cell cycle kinases are thermosensitive in vivo. The general validity of our kinant approach was demonstrated by our ability to create kinant mutants for all conserved cell cycle kinase families (i.e., Cdc5/PLK, Cdc7/DDK, Cdc15/Ste20-like kinase, Cdc28/CDK, and Ipl1/Aurora), across multiple species, including for example, mammalian and non-mammalian species.

Accordingly in one aspect there is provided a substantially pure nucleic acid encoding a eukaryotic protein kinase having at least one mutated amino-acid residue located in its subdomain IX.

In one example, the nucleic acid encodes a protein kinase sequence in which at least one mutated residue is the conserved aspartate or the residue immediately C-terminal to the conserved glycine position in the subdomain IX. The nucleic acid encodes an amino acid residue corresponding to D263 or V269 in Cdc5. The nucleic acid encodes a protein kinase polypeptide sequence in which the at least one mutation is selected from the group consisting of: N and Q for the conserved asparate position in the subdomain IX. The nucleic acid encodes a protein kinase polypeptide sequence in which the at least one mutation is selected from the group consisting of D and E for the residue immediately C-terminal to the conserved glycine position in the subdomain IX.

In another example, the nucleic acid encodes a protein kinase polypeptide sequence in which the mutations generate conditional temperature sensitive alleles.

In another example, the nucleic acid encodes a protein kinase polypeptide sequence in which the mutated eukaryotic protein kinase encode a polypeptide sequence selected from the group consisting of: SEQ ID NO: 1; SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; and SEQ ID NO: 12.

In one example, the nucleic acid is DNA which is operably linked to regulatory sequences for expression of the mutated eukaryotic protein kinase and wherein the regulatory sequences comprise a promoter. The promoter is a constitutive promoter or a promoter inducible by one or more external agents, or is cell-type specific.

In one example, the kinase is selected from the group consisting of the following families of kinases: AMPK, Aurora kinases, BUB1, CAK1, Cyclin-dependent kinases (CDKs) Calmodulin kinase, Casein kinases 1 and 2, CHK1, CHK2, MAP kinases, MAP kinase kinase, MPS1, NDR, and p21-activated kinase.

According to another aspect, there is provided a method of producing a mutated eukaryotic protein kinase, the method comprising:
a) transfecting a cell with a nucleic acid sequence, according to any one of claims 1 through 10 above, the nucleic acid encoding the mutated eukaryotic protein kinase positioned for expression in the cell;
b) culturing the transfected cells under conditions for expressing the nucleic acid;
c) producing the mutated eukaryotic protein kinase; and
d) temperature controlling the activity of the produced mutated eukaryotic protein kinase in the transfected cells.

According to yet another aspect, there is provided a substantially pure eukaryotic protein kinase polypeptide having a mutation in its subdomain IX, the kinase being encoded by the nucleic acid, as described above.

In one example, the polypeptide includes at least one mutant descriobed above.

In one example, the polypeptide or the nucleic acid, as described above, is non-mammalian. In one example, the non-mammal is a yeast strain.

In another example, the polypeptide or the nucleic acid, as described above, is mammalian.

According to another aspect, there is provided a vector comprising a nucleic acid, as described above, the vector being capable of directing expression of the polypeptide encoded by the nucleic acid n a vector-containing cell.

According to another aspect, there is provided a cell comprising a nucleic acid, as described above. In one example, the cell is from a eukaryotic organism.

According to another aspect, there is provided a method for controlling the enzymatic activity of a mutated eukaryotic kinase in a cell, the method comprising:
a) introducing at least one mutated residue into the subdomain IX of the kinase; and
b) subjecting the cell to changes in an external environmental parameter so as to control the enzymatic activity of the mutated eukaryotic kinase.

In one example, control of the enzymatic activity includes conditionally inactivating the mutated kinase polypeptide using changes in the external temperature of the cell. In one example, the external temperature is in the range of about 37° C. to about 41° C.

According to yet another aspect, there is provided a method of determining the physiological and pathological functions of a kinase in eukaryotic cells, the method comprising:
a) introducing at least one mutated residue ink) either of the subdomain IX;
b) subjecting the cells to changes in an external environmental parameter;
c) measuring the physiological and pathological functions of a WT kinase; and
d) comparing the physiological and pathological functions of the WT kinase with the mutated kinase so as to determine the physiological and pathological functions of the kinase.

According to still another aspect, there is provided a method for determining the minimal level of kinase activity required to execute a specific cellular function in eukaryotic cells, the method comprising:
a) introducing at least one mutated residue into either of the subdomain IX;
b) subjecting the cells to changes in an external environmental parameter;
c) measuring the quantitative requirement of a WT kinase; and
d) measuring the biochemical or cellular effects of progressively reducing the activitity of the mutated kinase using a series of increasing temperatures so as to determine the minimal level of kinase activity required to execute a specific cellular function.

According to another aspect, there is provided a method of determining whether a mammalian kinase is a therapeutic target for pharmaceutical intervention, the method comprising:
a) introducing at least one mutated residue into the subdomain IX of the kinase;
b) determining if the mutated kinase has reduced enzymatic activity compared to a WT kinase; and
c) determining that this reduced activity has an effect on cells that may be associated with a therapeutic benefit in a the context of a human disease, the effect resulting from the reduced enzymatic actvity of the mutated kinase being an indication that the kinase is a therapeutic target for pharmaceutical intervention.

According to yet another aspect, there is provided a method of quantitatively controlling protein kinase activity, the method comprising:
a) conferring thermosensitivity on the kinase by introducing at least one mutated residue into the subdomain IX of the kinase, the thermosensitivity of the kinase being used to modulate the enzymatic activity of the protein kinase at a specific stage in a cell cycle.

In one example, the method is carried out in vitro. In another example, the method is carried out in vivo.

### BRIEF DESCRIPTION OF THE DRAWINGS

In order that the invention may be readily understood, embodiments of the invention are illustrated by way of example in the accompanying drawings.

FIG. 1. Identification of cdc5 mutants defective in kinase function.

(A) Dilution series of wt cells and cdc5 mutants were spotted on YEPD plates to evaluate growth at various temperatures. (B) Multiple local alignment of the sequence of the F-helix region in selected cell cycle kinases. (C) Model structure of the kinase domain of Saccharomyces cerevisiae Cdc5 showing the position of the conserved F-helix Asp263 in purple. Selected hydrogen bonds linking Asp263 in the F-helix with key conserved residues in the catalytic loop of the kinase domain are displayed by dashed lines (10). (D) Model structure of the Cdc5 mutant carrying Gln263 in its F-helix. (E) Five-fold dilution series of yeast cells carrying mutations at position 1 and 2 of the F-helix of essential cell cycle kinases were spotted on YEPD plates to evaluate cell growth at various temperatures.

FIG. 2. Engineered mutants of Cdc5 are thermosensitive kinases in vitro and stable proteins in vivo.

(A) Kinase reactions were performed with purified Cdc5 and Cdc5-77 at various temperatures. Phosphorylated casein (pCasein) was resolved by electrophoresis and ³²P incorporation visualized by autoradiography (left). Quantification of the relative kinase activity of Cdc5 and Cdc5-77 at different temperatures is shown in the graph on the right. Kinase activity was normalized to the activity measured at 30° C. Error bars indicate SEM over three measurements. (B) Absolute kinase activity of the Cdc5-77 mutant. All kinase reactions contained identical amounts of Cdc5, Cdc5-77, or Cdc5 kinase-dead (short exposure autoradiogram: top panel; long exposure autoradiogram: middle panel; coomassie-stained gel: bottom panel). (C) Pre-activation of Cdc5 by Cdk1 does not reactivate Cdc5-77 kinase. Assays were performed as above except that Cdc5 was activated with Cdk1/cyclin B prior to conducting kinase assays. The weak background signal in all lanes for casein is due to the inefficient phosphorylation of this substrate by Cdk1/cyclin B (14). (D) Protein levels of various cdc5 mutants at restrictive temperature. Cells were synchronized in metaphase with nocodazole and incubated at 38° C. for two hours prior to protein extraction and analysis of Cdc5 levels by western-blotting. (E) Inactivation of SAN1 does not suppress the is phenotype of cdc5-77. Five-fold dilution series of single and double mutant were spotted on YEPD plates to evaluate cell growth at 22° C. and 37° C.

FIG. 3. Cell cycle phenotypes of cdc5-77 mutants.

cdc5-77 mutants together with the control cdc15-2 strain were arrested in G1 with α-factor and released from their arrest into a synchronous cell cycle at 38° C. Samples of cells were taken at the indicated time points to evaluate (A) budding index and mitotic spindle length, (B) cellular DNA content, and (C) cell morphology at the end of the experiment. (D) cdc15-2 and cdc5-77 mutants were synchronized as above and samples were taken at the indicated time points for analysis of phosphorylation-induced gel retardation of various Cdc5 substrates. For the analysis of Mcd1/Scc1 phosphorylation, cells were released from a G1 block into nocodazole-containing medium because this protein is degraded at the metaphase-anaphase transition (46, 47). Budding indexes (BI) are shown below gels. (E) Analysis of Cdc14 release from the nucleolus in wt, cdc15-2, and cdc5-77 mutants. Cells were synchronized and released as described above. (F) Mitotic spindle position relative to the mother-bud axis was determined at anaphase onset in the cells described in (E).

FIG. 4. (A) cdc5 mutants have different phenotypes. A schematic representation of the functional domains in Cdc5 is shown on the left, whereas selected phenotypes associated with various cdc5 alleles are shown in the table on the right. The PBD is made of three distinct elements : the Polo-cap (Pc) and Polo boxes 1 and 2 (PB1 and PB2). The position of known mutations in cdc5 mutants are depicted to scale on the protein schematic, along with the domains affected by the mutation(s). Phenotypes that are different between various alleles include: Bfa1 and Bub2 phosphorylation during mitotic exit (cdc5-1 vs cdc5-2 (1)); Cdc14 release from the nucleolus in early anaphase (cdc5-4 vs cdc5-1 and cdc5-10 (2-5)); formation of septin ring structure in cytokinesis (cdc5-1 and cdc5-3 vs cdc5-4 and cdc5-7 (6)); and adaptation to DNA damage (cdc5-ad vs cdc5-1 (7)). In the table on the right, phenotypes associated with each mutant alleles are annotated with ‘−’ if the specific allele shows a fully defective phenotype, ‘+’ if the allele shows no detectable defect for this phenotype, or ‘+/−’ for a partial defect of the allele for this phenotype (n.d.: phenotype not determined for this allele). The growth of various mutants under permissive conditions was also included in the table. Note that cdc5-1, cdc5-2, and cdc5-as1 mutants have significant growth defects even in the absence of temperature challenge or inhibitor (8, 9). Finally, the stability of Cdc5 protein at restrictive temperature is included in the table (‘+’ means normal stability for mutant protein, means completely unstable mutant protein, ‘+/−’ means partially unstable mutant protein, and ‘++’ means higher than normal levels) (6, 10). (B) Genetic analysis of sporulated heterozygous diploid strains carrying a kinase-inactivating point mutation, K110M, in Cdc5 kinase subdomain II. The genotype of the resulting segregants was deduced using the HIS3 marker associated with cdc5 mutation. None of the cells that germinate after sporulation carry the kinase-inactivating mutation in CDC5. (C) Cdc5 protein levels in wt and cdc5-as1 mutant yeast. Protein extracts were prepared from CDC5 and cdc5-as1 strains and analyzed for Cdc5 levels by western-blotting using a monoclonal antibody against Cdc5. Nsp1 protein levels are used as loading control. (D) Inactivation of SAN1 suppresses the is phenotype of cdc5-1. Five-fold dilution series of single and double mutants were spotted on YEPD plates to evaluate cell growth at 22° C. and 30° C. Triangles indicate decreasing concentration of cells in spots. Please note the suppression of the lethality of cdc5-1 mutant by SAN1 deletion at 30° C. Cdc5-1 is known to be a partially unstable protein at non-permissive temperature (see FIG. 2D and reference (6)). (E) Cdc15 protein levels in wt and cdc15-85 mutant. Protein extracts were prepared from strains incubated at either 22° C. or 37° C. Protein levels of MYC-tagged Cdc15 were monitored by western-blotting using a monoclonal antibody against the MYC epitope. Pgk1 was used as a loading control.

FIG. 5. (A) cell cycle analysis of cdc5-77 mutants. cdc5-77 mutants together with wt controls were arrested in G1 with α-factor and released from their arrest into a synchronous cell cycle at 38° C. Samples of cells were taken at the indicated time points to evaluate budding index and mitotic spindle length. (8) cdc5-77 and SLK19-3xHA are synthetic lethal. The genotype of segregants isolated from a sporulated heterozygous diploid strain carrying cdc5-77 and SLK19-3xHA was deduced using the HIS3 marker associated with cdc5-77 and the TRP1 marker associated with SLK19-3xHA. (C) Double inactivation of Cdc5 kinase and PBD activities leads to lethality. Genetic analysis of a heterozygous diploid strain carrying the cdc5-20 allele affecting both the kinase activity (D263Q≈cdc5-77) and PBD activity (W517F-H641A-K643M cdc5-16) of Cdc5. Note that cdc5-20 allele is linked to a HIS3 marker. Finally, I would like to mention that the proof-of-concept that kinants can be used in “synthetic-lethal” genetic screens is made in FIG. S2B. Specifically, we show in this figure that cdc5-77::HIS3MX6 mutants are lethal when combined to a SLK19-3HA::TRP1 mutant (whereas both individual mutants are viable and grow well under standard growth conditions).

FIG. 6. The experiment shown in this Figure demonstrates that a kinase from another organism than budding yeast is susceptible to the kinant approach. In particular, we demonstrate in this Figure that the kinant “1” versions of the polo kinase from Drosophila melanogaster (both the “D→Q” and the “D→N” versions) can complement a polo-like kinase mutant in yeast (i.e., cdc5-1) in a temperature-sensitive manner. This type of assay is called a “trans-species complementation assay”. The Figure shows that the DQ and DN mutants of polo kinase are capable of growing at 30° C. but not at 37° C. (the kinant 2 “C→D” mutant should be used as a negative control/baseline in this experiment).

FIG. 7 is a Table showing examples of yeast strain used containing mutated (and thus mutable) eukaryotic kinases: D1032; D1224; D1640; D1799; D979; D1988; D2010; D1964; D140; D1015.

### DETAILED DESCRIPTION

In the following description of the embodiments, references to the accompanying drawings are by way of illustration of an example by which the invention may be practiced. It will be understood that other embodiments may be made without departing from the scope of the invention disclosed.

Definitions:

Unless otherwise specified, the following definitions apply throughout:

As used herein, the singular forms “a”, “an”, and “the” include plural referents unless the context clearly indicates otherwise. Thus, for example, reference to “a mutation” includes one or more of such mutations and reference to “the method” includes reference to equivalent steps and methods known to those of ordinary skill in the art that could be modified or substituted for the methods described herein.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “eukaryotic protein kinase” is intended to refer to a protein, a polypeptide or fragment thereof, encoded by a eukaryotic protein kinase gene. Examples of wild-type (WT) eukaryotic protein kinases include cyclin-dependent kinase, polo-like kinase 1, Aurora B kinase, and Cdc7 kinase.

As used herein, the term “mutated eukaryotic protein kinase” is intended to mean a (Wild-Type) (WT) eukaryotic protein kinase in which one or more amino acid residues in the protein sequence have been changed. In certain examples described herein, the mutations include D263N, V269D, D263Q and V269E, which are located in the subdomain IX of the yeast polo kinase Cdc5. More generally, the mutations described herein are located in the subdomain IX of eukaryotic protein kinases. The mutated residues affect the conserved aspartate in subdomain IX or the residue immediately C-terminal to the conserved glycine position in subdomain IX. Also contemplated in this discovery are kinases selected from the group consisting of, but not limited to: mammalian AMPK, BUB1, CAK1, Calmodulin kinase, Casein kinases 1 and 2, CHK1, CHK2, MAP kinases, MAP kinase kinase, MPS1, NDR, and p21-activated kinase.

As used herein, the term “mutation” is intended to mean any alteration in a gene which alters function or expression of the gene products, such as mRNA and the encoded for protein.

As used herein, the term “eukaryotic protein kinase gene” is intended to mean a gene encoding a eukaryotic protein kinase having a subdomain IX. The eukaryotic protein kinase protein gene is a gene having about 50% or greater amino acid sequence identity to conserved kinase features, in particular the 12 subdomains defined as “regions never interrupted by large amino acid insertions and containing characteristic patterns of conserved residues”, as initially described by Hanks and Hunter (1995) Faseb Journal 9:576-796. For example, subdomain IX was originally defined as consensus sequence: “Doo+ogoooo-o”. This consensus is given according to the following code: uppercase letters, invariant residue; lowercase residues, nearly invariant residues; o, positions conserving nonpolar residues; +, positions conserving small residues with near neutral polarity (taken from Hanks and Hunter [1995]).

As used herein, the term “quantitative requirement” when used in reference to a kinase, is intended to mean the specific level (e.g., 20, 30, 40%) of kinase activity required to execute a specific cellular function of a kinase. This is relevant because most kinases perform multiple distinct tasks in a cell and not all tasks require the same “quantitiy” or level of kinase activity to be completed.

As used herein, the term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein and is generally found between exons.

As used herein, the term “subdomain IX” is intended to mean a site in the WT eukaryotic protein kinase polypeptide sequence or the mutated eukaryotic protein kinase polypeptide sequence, which act as a structural anchor for several regulatory and structural residues within kinase domain. In particular, subdomain IX (also known as F-helix by experts), is believed to act as an organizing “hub” providing precise positioning to key catalytic and regulatory elements (Kornev, Taylor and Ten Eyck [2008] PNAS vol. 105, pages 14377-14382).

As used herein, the term “detectable label” is intended to mean a compound that may be linked to a eukaryotic protein kinase subdomain IX, such that when the compound is associated with the subdomain, the label allows either direct or indirect recognition of the compound so that it may be detected, measured and quantified.

As used herein, the term “affinity tag” is intended to mean a ligand or group, which is linked to a eukaryotic protein kinase subdomain IX to allow another compound to be extracted from a solution to which the ligand or group is attached.

As used herein, the term “nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids described herein, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Whenever applicable, the term “isolated nucleic acid” may also refer to a RNA molecule encoded by an isolated DNA molecule as defined above. Alternatively, the term may refer to an RNA molecule that has been sufficiently separated from other nucleic acids with which it would be associated in its natural state (i.e. in cells or tissues). An “isolated nucleic acid” (either DNA or RNA) may further represent a molecule produced directly by biological or synthetic means and separated from other components present during its production.

As used herein, the term “vector” is intended to mean a replicon, such as a plasmid, cosmid, bacmid, phage or virus, to which another genetic sequence or element (either DNA or RNA) may be attached so as to bring about the replication of the attached sequence or element.

As used herein, the terms “percent similarity”, “percent identity” and “percent homology” when referring to a particular sequence are used as set forth in the University of Wisconsin GCG software program.

As used herein, the term “substantially pure” is intended to refer to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like). Described herein are substantially pure mutated eukaryotic protein kinases (e.g., nucleic acids, oligonucleotides, proteins, fragments, mutants, etc.).

As used herein, the term “oligonucleotide” is intended to sequences, primers and probes as described herein, and is defined as a nucleic acid molecule comprised of two or more ribo- or deoxyribonucleotides, preferably more than three. The exact size of the oligonucleotide will depend on various factors and on the particular application and use of the oligonucleotide.

As used herein, the term “primer” is intended to refer to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic appications, the oligonucleotide pririer is typically about 20-40, or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

As used herein, the term “probe” is intended to refer to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains about 20-40 or more nucleotides in length, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

With respect to single-stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule as described herein, to the substantial exclusion of hybridization of the oligonucleotide with single-stranded nucleic acids of non-complementary sequence. Appropriate conditions enabling specific hybridization of single-stranded nucleic acid molecules of varying complementarity are well known in the art. For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):

Tₘ=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5 with every 1% decrease in homology. Thus, targets with greater than about 75% sequence identity would be observed using a hybridization temperature of 42° C.

The stringency of the hybridization and wash depends primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. With regard to the nucleic acids as described herein, a moderate stringency hybridization is defined as hybridization in 6×SSC, 5×Denhardt's solution, 0.5% SDS and 100 μg/ml denatured salmon sperm DNA at 42° C. and washed in 2×SSC and 0.5% SDS at 55° C. for 15 minutes. A high stringency hybridization is defined as hybridization in 6×SSC, 5×Denhardts solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

Alternatively, as used herein, the term “probe” is intended to mean a compound which is labeled with either a detectable label or an affinity tag, and which is capable of binding, either covalently or non-covalently, to a eukaryotic protein kinase protein subdomain IX. When, for example, the probe is non-covalently bound, it may be displaced by a test compound. When, for example, the probe is bound covalently, it may be used to form cross-linked adducts, which may be quantified and inhibited by a test compound.

As used herein, the term “isolated protein” or“isolated and purified protein” is intended to refer to a protein produced by expression of an isolated nucleic acid molecule as described herein. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

As used herein, the term “amino acid” is intended to mean a radical derived from the corresponding a-amino acid by eliminating the hydroxyl of the carboxy group and one hydrogen of the .alpha.-amino group. For example, the terms Gln, Ala, Gly, Ile, Arg, Asp, Phe, Ser, Leu, Cys, Asn, and Tyr represent the residues of L-glutamine, L-alanine, glycine, L-isoleucine, L-arginine, L-aspartic acid, L-phenylalanine, L-serine, L-leucine, L-cysteine, L-asparagine, and L-tyrosine, respectively. Amino Acid residues are provided below:

Three and single letter abbreviations for a-amino acids used throughout are as follows:

| Amino acid. | Abbreviation | Abbreviation |
|---|---|---|
| Alanine | Ala | A |
| Arginine | Arg | R |
| Aspartic acid | Asp | D |
| Asparagine | Asn | N |
| Cysteine | Cys | C |
| Glutamic acid | Glu | E |
| Glutamine | Gln | Q |
| Glycine | Gly | G |
| Isoleucine | Ile | I |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “solid support” refers to any solid or stationary material to which reagents such as antibodies, antigens, and other test components can be attached. Examples of solid supports include, without limitation, microtiter plates (or dish), microscope (e.g. glass) slides, coverslips, beads, cell culture flasks, chips (for example, silica-based, glass, or gold chip), membranes, particles (typically solid; for example, agarose, sepharose, polystyrene or magnetic beads), columns (or column materials), and test tubes. Typically, the solid supports are water insoluble.

We were prompted to develop the kinant approach because no kinase-specific mutation was available to study Cdc5 kinase (yeast polo kinase; the homolog of the cancer-associated PLK1 kinase in human). In particular, the previously-described “analog-sensitive” approach used to inactivate protein kinases does not work with Cdc5 (˜30% of kinases are resistant to this approach; Zhang et al., 2005). Cdc5, like all PLK family members, is a multidomain kinase containing a polo-box domain (PBD). We wanted to determine whether the kinase domain and PBD of Cdc5 have the same or different functions during cell cycle progression. Our ability to dissociate the kinase and PBD activities of Cdc5 allowed us to identify completely novel fonctions for this conserved family of cancer-associated kinases. Taken together, our discoveries support strongly the feasibility and rationale of using kinants as tools to unravel novel kinase-specific functions in conserved cancer-associated kinases.

Modification of proteins by reversible phosphorylation is a major regulatory mechanism in eukaryotic cells. In particular, progression through the cell division cycle depends largely on regulated protein phosphorylation. Consistent with this, cell cycle kinases are the central effectors of cell proliferation in all eukaryotes. These kinases include cyclin-dependent kinases (CDKs), polo-like kinases (PLKs), aurora kinases, and Cdc15 family of kinases. We therefore developed a completely novel approach to control kinase activity in vivo using engineered mutations in multiple cell cycle kinases. The engineered kinases created by this new approach—which we refer to as “kinants”—enables us to examine the specific cell cycle functions of protein kinases in ways that were not possible in the past.

I. Nucleic Acids, Polypeptides, Cells and Vectors

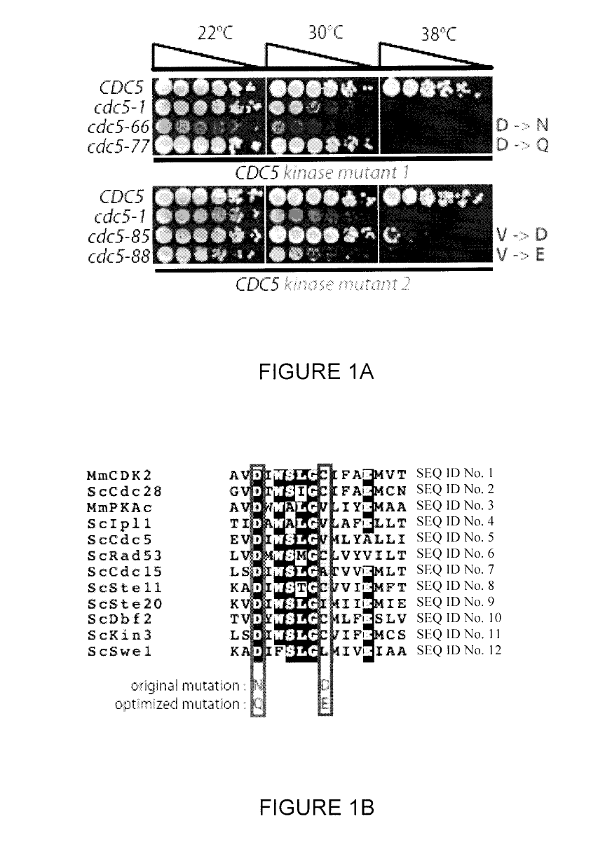

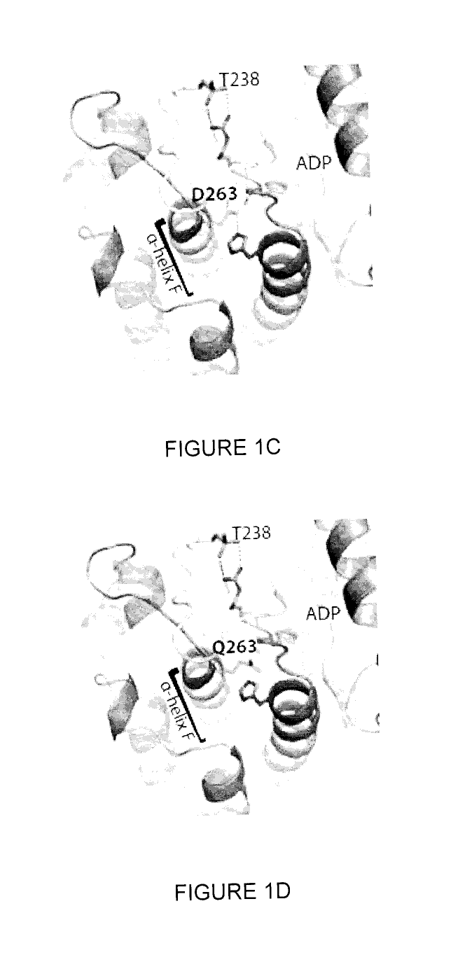

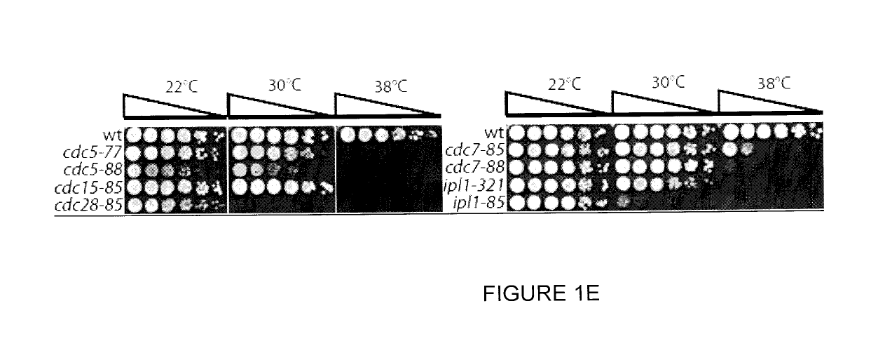

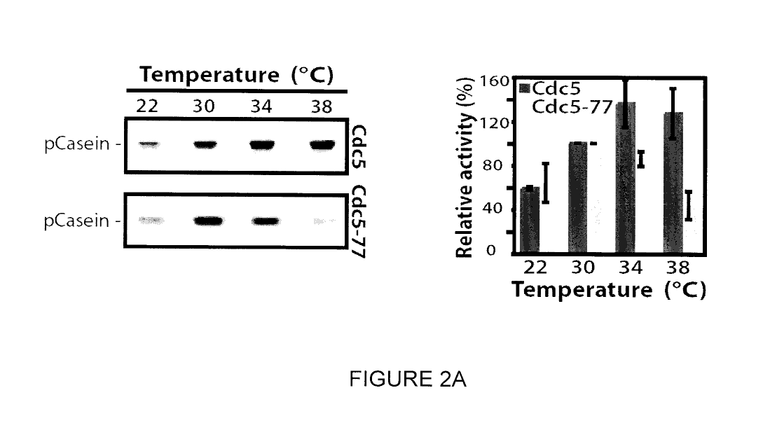

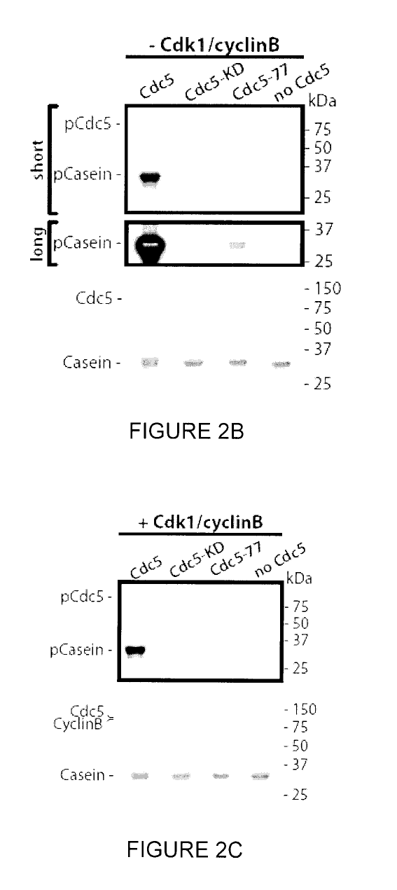

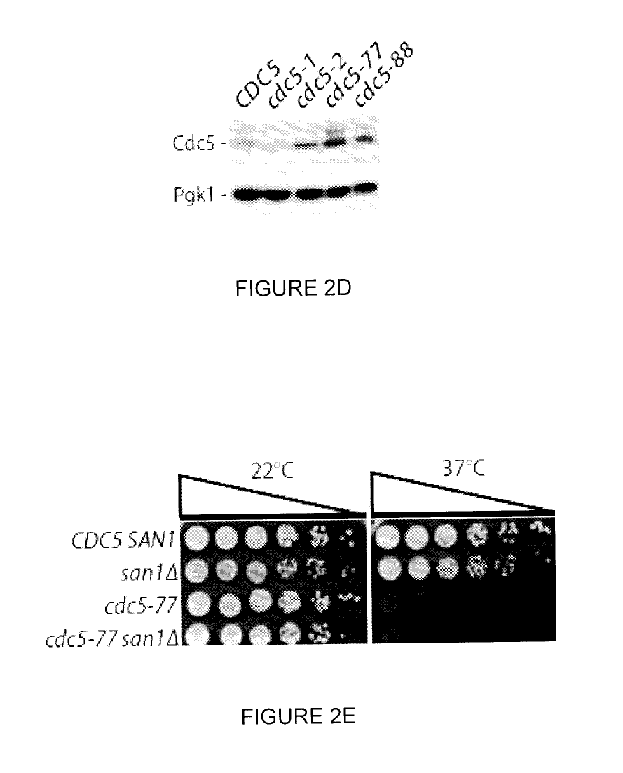

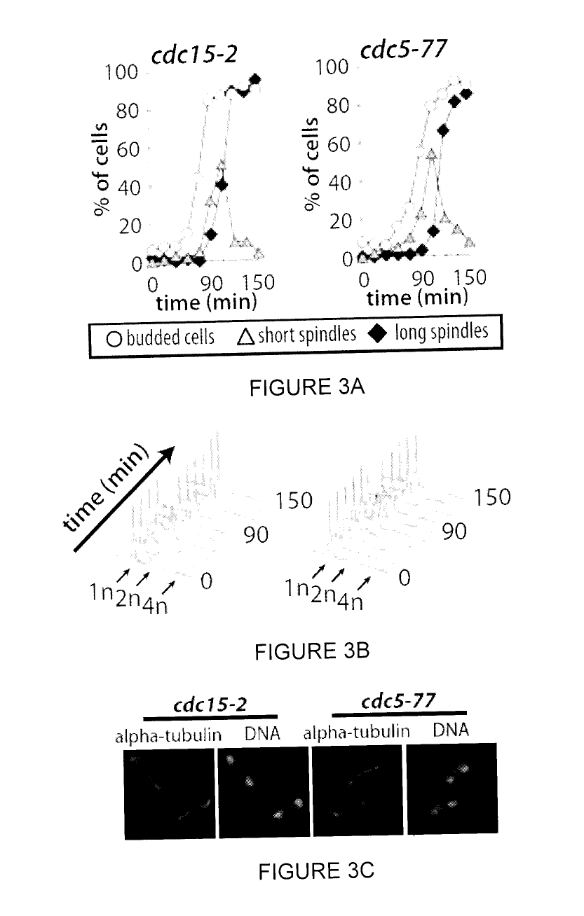

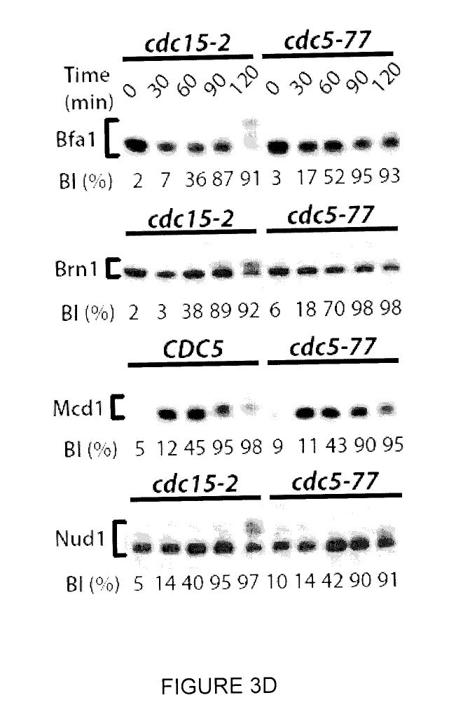

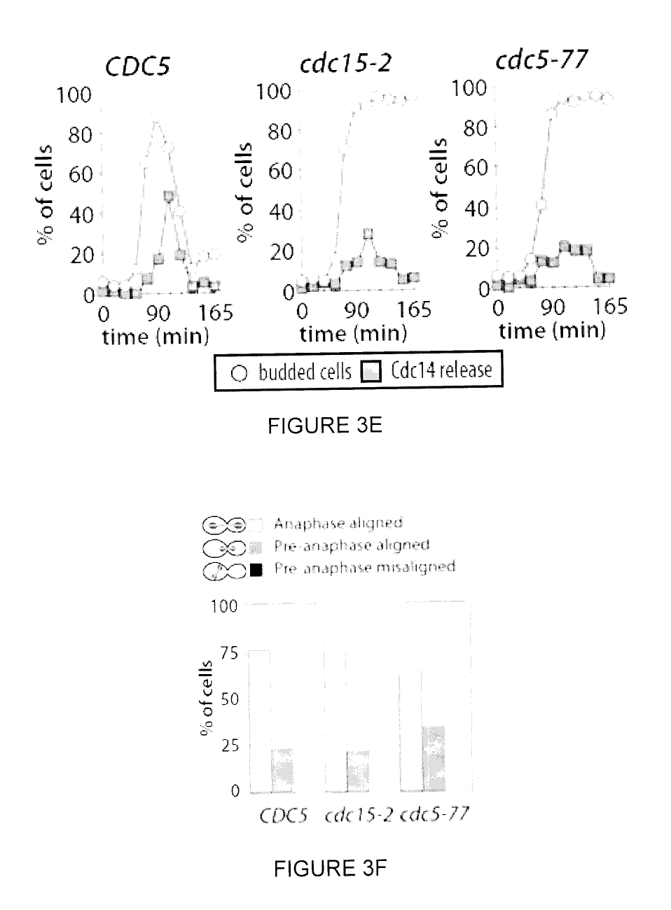

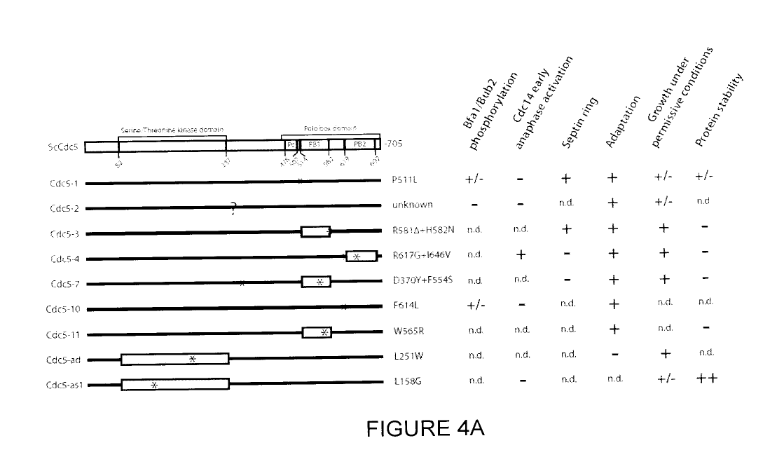

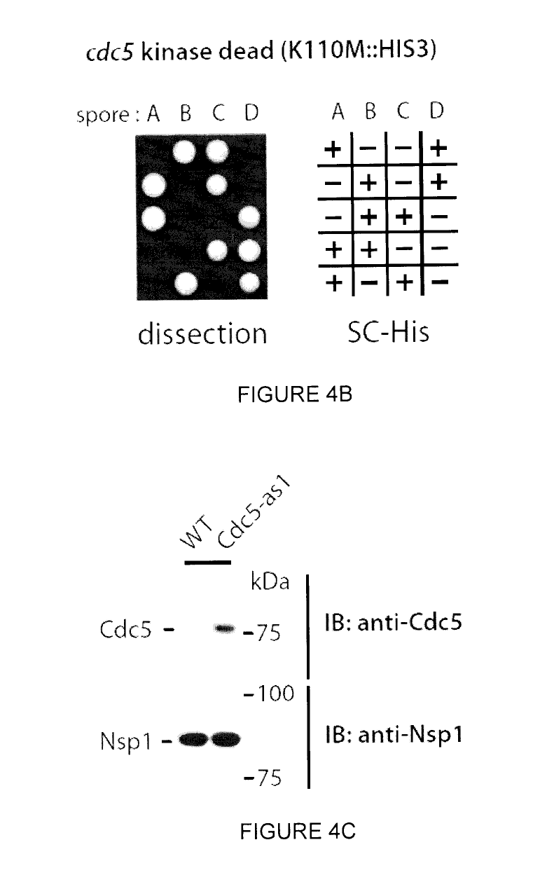

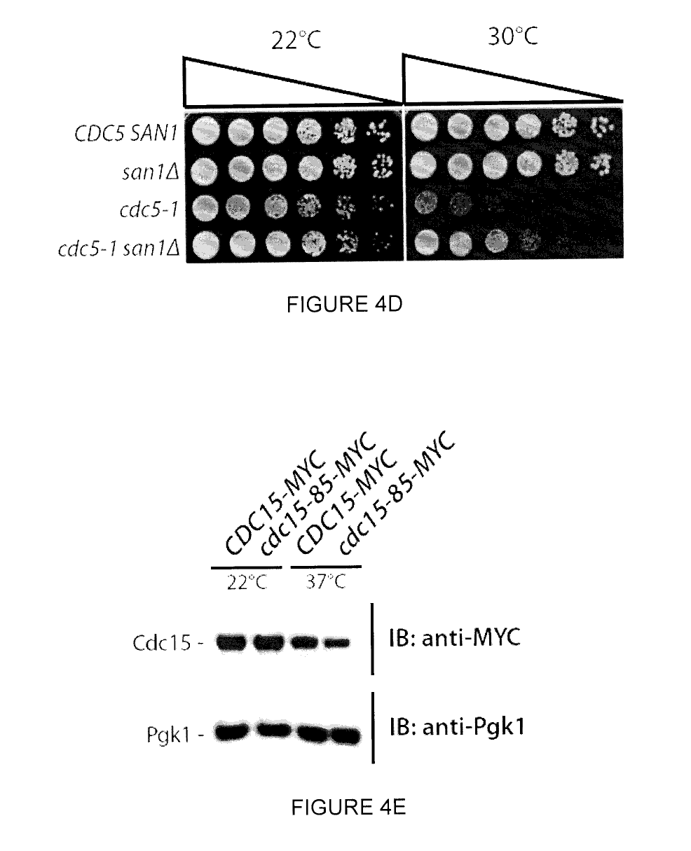

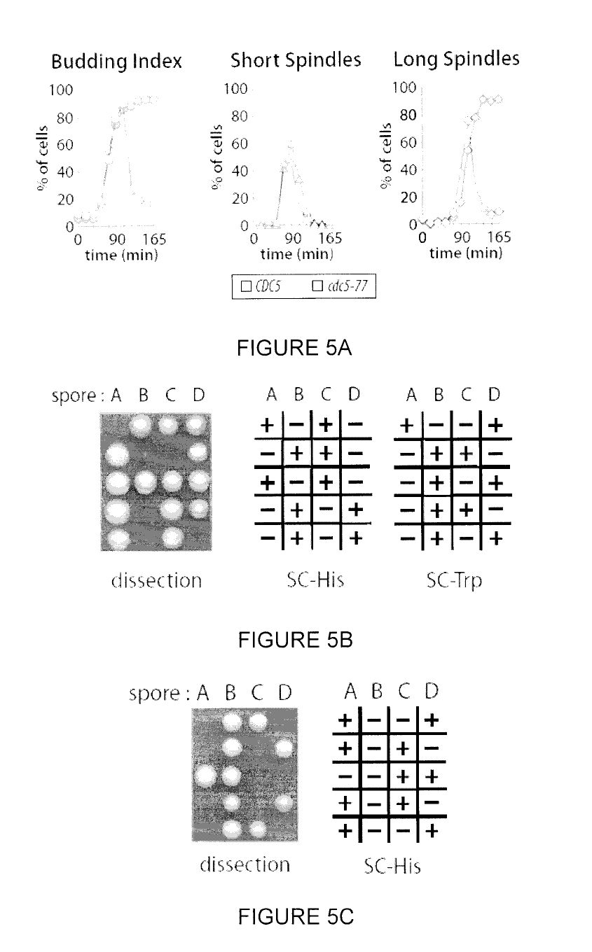

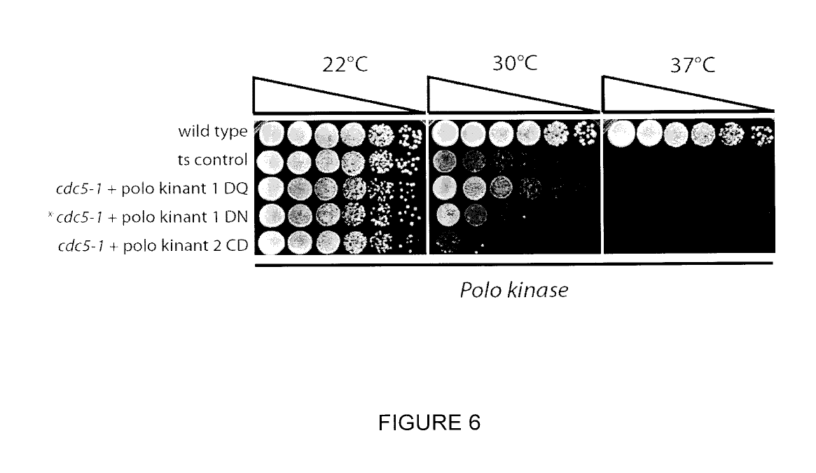

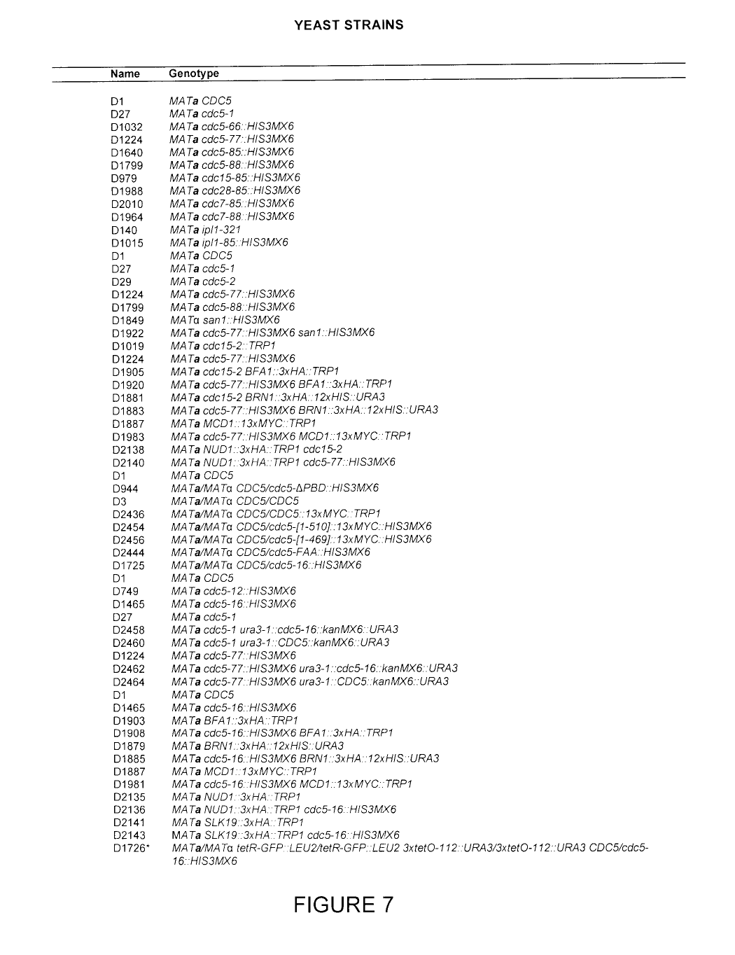

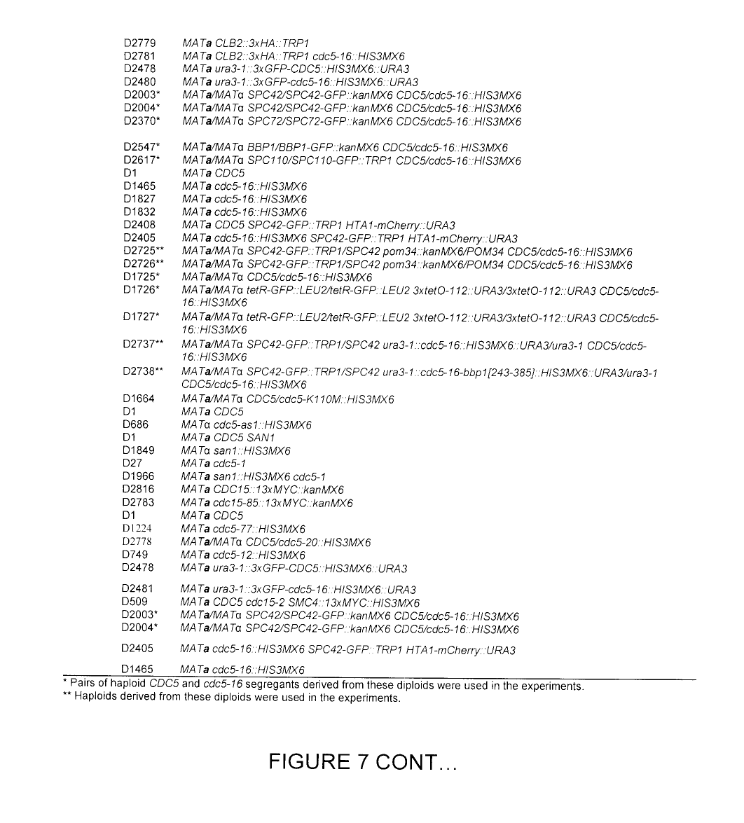

We have now discovered a substantially pure nucleic acid encoding a eukaryotic protein kinase, which has at least one mutated amino acid residue located in its subdomain IX. The at least one mutated residue is the conserved aspartate or the residue immediately C-terminal to the conserved glycine position in the subdomain IX. These residues correspond to D263 or V269 in Cdc5. The mutation is selected from the group consisting of: N and Q for the conserved asparate position in the subdomain IX. The mutation is selected from the group consisting of D and E for the residue immediately C-terminal to the conserved glycine position in the subdomain IX. The mutations generate conditional temperature sensitive alleles. Referring to FIG. 1B, the mutated eukaryotic protein kinase includes a sequence selected from the group consisting of: SEQ ID NO: 1; SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; and SEQ ID NO: 12. The nucleic acid is DNA which is operably linked to regulatory sequences for expression of the mutated eukaryotic protein kinase and wherein the regulatory sequences comprise a promoter. The promoter is a constitutive promoter or a promoter inducible by one or more external agents, or is cell-type specific.

A substantially pure eukaryotic protein kinase polypeptide having a mutation in its subdomain IX. The kinase is encoded by the nucleic acids described above. The polypeptide includes at least one mutant, which correspond to D263 or V269 in Cdc5. The mutation is selected from the group consisting of: N and Q for the conserved asparate position in the subdomain IX. The mutation is selected from the group consisting of D and E for the residue immediately C-terminal to the conserved glycine position in the subdomain IX. In one example, the polypeptide or the nucleic acid is non-mammalian, such as yeast strain as illustrated in FIG. 7.

In another example, the polypeptides or the nucleic acids described above are mammalian AMPK, BUB1, CAK1, Calmodulin kinase, Casein kinases 1 and 2, CHK1, CHK2, MAP kinases, MAP kinase kinase, MPS1, NDR, and p21-activated kinase.

Also contemplated is a vector which comprises a nucleic acid described above which is capable of directing expression of the polypeptide encoded by the nucleic acid in a vector-containing cell. Also contemplated is a cell, which comprises a nucleic acid in which the cell is taken from a eukaryotic organism.

II. Methods

In one aspect, a method of producing a mutated eukaryotic protein kinase is described in which the method comprises transfecting a cell with a nucleic acid sequence, which are described above. The nucleic acid encoding the mutated eukaryotic protein kinase is positioned for expression in the cells. The transfected cells are cultured under conditions for expressing the nucleic acid so as to produce the mutated eukaryotic protein kinase and then temperature controlling the activity of the produced mutated eukaryotic protein kinase in the transfected cells.

Alternatively, in another aspect, a method for controlling the enzymatic activity of a mutated eukaryotic kinase in a cell comprises introducing at least one mutated residue into the subdomain IX of the kinase; and subjecting the cell to changes in an external environmental parameter, typically temperature, but could also include the presence of unusual compounds in cell culture medium (such as, for example, formamide, dimethyl sulfoxide, heavy water (D₂O), tetracaine), or higher- than-normal concentrations of growth medium components (such as, for example, sodium chloride, ethanol) so as to control the enzymatic activity of the mutated eukaryotic kinase.

Controlling the enzymatic activity of the kinase includes conditionally inactivating the mutated kinase polypeptide using changes in the external temperature of the cell. Generally speaking, a typical range of non-permissive temperature for kinant mutants in eukaryotic organisms would be between about 37° C. to about 41° C., although different temperatures may also work.

Another method of determining the physiological and pathological functions of a kinase in eukaryotic cells comprises introducing at least one mutated residue into either of the subdomain IX, as described above and subjecting the cells to changes in an external environmental parameter. The physiological and pathological functions of a WT kinase were measured; and by comparing the physiological and pathological functions of the WT kinase with the mutated kinase we are able to determine the physiological and pathological functions of the kinase.

In another method for determining the minimal level of kinase activity required to execute a specific cellular function (quantitative requirement for a kinase activity) in eukaryotic cells, the method comprises introducing at least one mutated residue into either of the subdomain IX, as descibed above and subjecting the cells to changes in an external environmental parameter. By measuring the quantitative requirement of a WT kinase and measuring the biochemical or cellular effects of progressively reducing the activity of the mutated kinase using a series of increasing temperatures, such as 1° C. increments from 27° C. to 41° C., we can determine the minimal level of kinase activity required to execute a specific cellular function. Kinases typically have more than one function in cells. Not all functions require the same level of kinase activity. This means that it is possible to genetically separate functions carried out by a single kinase by progressively reducing its activity and finding the threshold at which a given function cannot be executed but other functions of that kinase are still unaffected.

In an alternative method, we can determine whether a mammalian kinase is a viable therapeutic target for pharmaceutical intervention. This method comprises introducing at least one mutated residue into the subdomain IX of the kinase, as described above and determining if the mutated kinase has reduced enzymatic activity compared to a WT kinase. It is then possible to determine that this reduced activity has an effect on cells that may be associated with a therapeutic benefit in a the context of a human disease, the effect resulting from the reduced enzymatic activity of the mutated kinase being an indication that the kinase is a therapeutic target for pharmaceutical intervention. Such human disease may include, for example, cancer, diabetes, immune disorders, and neurological diseases and the like.

III. Regulation of Cdc5 Kinase Activity by F-Helix Mutations

We first performed a genetic screen to identify mutations affecting the F-helix of Cdc5 kinase domain. This helix plays an integrative role in eukaryotic protein kinases (EPKs) by acting as an anchor for distinct parts of the kinase domain (Kannan and Neuwald, 2005; Kornev et al., 2008). While this helix is conserved in all EPKs, we noticed that it is absent in atypical kinases (Kannan and Neuwald, 2005) and reasoned that it may not be absolutely required for catalysis. In light of this, we hypothesized that mutations in the F-helix may affect kinase activity in a moderate or conditional manner. To test this, we generated both random and targeted mutations in Cdc5 kinase domain. From this pool, we identified two specific mutations, D263N and V269D, that when introduced in the F-helix of Cdc5 generated temperature-sensitive (ts) alleles (FIGS. 1A, B). One of the mutations affects the F-helix aspartate conserved in all EPKs (D263) and prevents the formation of an important network of hydrogen-bonds with residues in the catalytic loop (Kannan and Neuwald, 2005) (H196, H202, R203; FIG. 1C). The other mutation affects valine 269, a residue involved in the formation of several conserved hydrophobic interactions important for kinase-substrate association (Kannan and Neuwald, 2005). Importantly, the severity of the phenotype of the original mutants could be modulated and optimized by changing the nature of the amino acid residue at the specific position affected in the original mutants (cdc5-77: D263Q and cdc5-88: V269E; FIG. 1A-D). Together, these results indicate that it is possible to engineer kinase-specific conditional mutations in Cdc5.

Our ability to create Cdc5 mutants with conditional kinase activity prompted us to ask whether other essential kinases might be engineered in a similar manner. Remarkably, introducing mutations corresponding to cdc5-77 and cdc5-88 in essential cell cycle kinases resulted in the creation of at least one novel conditional allele for each of these kinases (FIG. 1E). For example, engineering both positions in Ipl1 kinase resulted in two ts alleles, including a novel high sensitivity ts allele, ipl1-85. Interestingly, the previously published ipl1-321 and mps1-6 alleles carry mutations that affect the same kinase positions as those modified in cdc5-77 and cdc5-88, respectively (Biggins et al., 1999; Schutz and Winey, 1998). Collectively, our results indicate that the F-helix region of EPKs is a mutagenic hotspot to create useful conditional mutants in essential kinases in yeast.

We next sought to determine whether Cdc5 kinase mutations could modulate the enzymatic activity of the engineered protein. To this end, we purified wild-type (wt) Cdc5 and Cdc5-77 mutant, and performed kinase activity assays at various temperatures. As expected, the kinase activity of wt Cdc5 increased with temperature from 22° C. to 34° C. and remained high at 38° C. (FIG. 2A). In contrast, the kinase activity of Cdc5-77 started to diminish at temperatures above 30° C. and was significantly reduced at 38° C. (FIG. 2A). We needed to use much larger amounts of mutant relative to wt Cdc5 to accurately monitor phosphorylation in these kinase assays. To better compare the absolute difference in activity of those kinases, we performed additional experiments in the presence of equal amounts of wt and mutant Cdc5. Strikingly, we observed that even at the optimal temperature of 30° C., the kinase activity of Cdc5-77 is ≦1-2% of wt levels (FIG. 2B). Interestingly, cell survival in the presence of very low levels of kinase activity has been reported with several essential kinases (Hermosilla et al., 2005; Simanis and Nurse, 1986; Simizu and Osada, 2000). This low level of activity is not due to reduced phosphorylation of the activation loop of Cdc5 because pre-phosphorylation of the mutant by Cdk1 (Mortensen et al., 2005) did not improve its kinase activity (FIG. 2C; note that Cdk1 can phosphorylate casein weakly, thereby covering up the weaker Cdc5 mutant signal). Although the kinase activity of Cdc5-77 is thermosensitive in vitro, it remains a possibility that the conditional phenotype of cdc5-77 mutants is due in part to reduced protein stability in vivo. To address this, we directly monitored Cdc5 protein stability in mutant and wt cells. FIG. 2D shows that Cdc5-77 and Cdc5-88 protein levels were not reduced relative to wt Cdc5 at non-permissive temperature. Consistent with this result, inactivation of the protein quality control pathway (Gardner et al., 2005) did not suppress the ts phenotype of cdc5-77 mutants (FIGS. 2E and 4D). Together, these results indicate that the kinase activity of Cdc5-77 is thermosensitive in vitro and that its protein levels are not reduced at non-permissive temperature relative to wt Cdc5 in vivo.

IV. Loss of Cdc5 Kinase Activity Leads to Mitotic Exit Defects

The creation of mutants that specifically modulate the kinase activity of Cdc5 allowed us to investigate the specific roles of this activity in vivo. We first compared cell cycle progression of a synchronized culture of cdc5-77 mutants (i.e., kinase-defective Cdc5) with that of a control cdc15-2 mutant. We used a cdc15-2 mutant as control in this experiment because this strain arrest during mitosis at a stage similar to cdc5 mutants (Surana et al., 1993), which allows direct comparison of cell cycle progression between both strains. Interestingly, cdc5-77 mutants growing at non-permissive temperature progressed normally from G1 up to early mitosis but arrested with elongated mitotic spindles and a 2n DNA content (135 min and later; FIG. 3A-C). As expected for a Cdc5 kinase-defective strain, several proteins that normally become phosphorylated in mitosis—including Bfa1, Brn1, Mcd1/Scc1, and Nud1—showed little or no phosphorylation in cdc5-77 mutants (FIG. 3D). In contrast, those substrates show high levels of phosphorylation in cdc15-2 mutants arrested in late mitosis (FIG. 3D).

We next sought to better define the late mitotic defect of cdc5-77 mutants. One of the key functions of Cdc5 in anaphase is to regulate the release and activation of Cdc14 from its nucleolar inhibitor, Cfi1/Net1 (D'Amours and Amon, 2004). To address the role of Cdc5 kinase activity in this process, we compared Cdc14 nucleolar release in wt, cdc15-2, and cdc5-77 mutants released from α-factor arrest. It was necessary to include a wt strain in this experiment because cdc15-2 mutants are defective in Cdc14 release by the mitotic exit network (MEN; Stegmeier et al., 2002). Immunofluorescence analysis of those strains revealed that the nucleolar release of Cdc14 was significantly diminished in cdc5-77 mutants relative to wt (FIG. 3E). In fact, most cdc5-77 cells contained little-to-no Cdc14 released from the nucleolus once they reached the end of the time course experiment (FIG. 3E). This phenotype is very similar to the Cdc14 release defect seen in MEN-deficient mutants (i.e., cdc15-2; FIG. 3E) (Stegmeier et al., 2002). Collectively, our results indicate that cdc5-77 mutants are at least partially competent in Cdc14 activation by the FEAR network, but completely defective in the activation of Cdc14 mediated by the MEN. Mechanistically, this mitotic exit defect can be explained by absence of phosphorylation of key MEN regulators—Bfa1 and Nud1—in cdc5-77 cells (FIG. 3D).

Finally, we wanted to test the role of Cdc5 in spindle positioning in mid-mitosis, since it was shown recently that this function was defective in a specific cdc5 mutant (Snead et al., 2007). The relative localization of the mitotic spindle was compared to the mother-bud axis at anaphase onset in cdc5-77 mutants and control cells, as previously described. We did not detect any increase in misaligned spindles in cdc5-77 mutants relative to control strains (FIG. 3F). Taken together, these results show that the execution of late mitotic processes is acutely sensitive to the levels of Cdc5 kinase activity. Even though clear defects in Cdc5 substrate phosphorylation are visible prior to anaphase in cdc5-77 mutants, their most striking cellular phenotype is a complete block in the execution of mitotic exit.

Using the kinant technology described above, we were able to successfully discriminate between the cellular functions of the kinase and polo box domains in the Cdc5 kinase (see Ratsima et al in PNAS vol. 108 (43) E914-E923 for further details).

Discussion

We have demonstrated that the functions of the kinase domain and PBD of Cdc5 can be dissociated genetically with strikingly different consequences for cells. This dichotomy is particularly evident in the effects of PBD inactivation on cellular ploidy. Indeed, this phenotype is only seen in PBD mutants and unravels a novel function for Cdc5 as an important regulator of cellular ploidy. Our ability to successfully separate the phenotypes of the kinase and PBD domains of Cdc5 highlights the benefit of examining mutants affected in specific biochemical activities in order to obtain an integrative view of the roles of multifunctional kinases in vivo. Consistent with this, PBD-specific mutants of Cdc5 have previously been very useful in the identification of novel functions for PLKs (27).

At a cellular level, one of the most striking defects in Cdc5-16 mutants is their inability to incorporate and/or maintain all SPB components in fully functional MTOCs. Multiple lines of evidence support a direct functional relationship between impaired regulation of SPB components and ploidy defects in Cdc5-16 mutants. First, loss of ploidy control is frequently observed in mutants defective in the regulation of SPB components (e.g., 12). Second, multiple components of the SPB are regulated by Cdc5-mediated phosphorylation (22, 32, 33). Third, Cdc5 localizes to SPBs (6, 24) and interacts physically with multiple SPB components (22, 33, 34). Fourth, mutants of SPB outer plaque components that are known to physically interact with Cdc5—namely Spc72 and Cnm67 (22, 34)—show an uncommon age-dependent increase in ploidy that is reminiscent of the progressive changes in ploidy experienced by Cdc5-16 cells (35). Finally, the specific constellation of cytological defects seen in cdc5-16 mutants (i.e., supernumerary Spc42/Spc72 structures, reduced functionality of SPBs, and aberrant numbers of astral microtubules) is also associated with loss of ploidy control in mutants affecting the SPB components NDC1 and CNM67 (36-38). Collectively, these observations indicate that Cdc5 plays an important role in the regulation of SPB function and in ploidy control.

Our results are consistent with previous studies implicating PLKs in the regulation of centrosome maturation in metazoans (4, 39). In particular, the mislocalization of a number of SPB components in cdc5-16 mutants is reminiscent of the inability of human and Drosophila cells to effectively localize γ-tubulin-containing complexes to centrosomes in the absence of polo kinase activity (4, 39). This defect is also associated with loss of ploidy control in cells derived from polo mutant flies, similar to what we observe with cdc5-16 mutants in yeast (40). Moreover, the presence of aberrant assemblies of centrosome/SPB components in both Plk1- and Cdc5-defective cells highlights the phenotypic similarity between these two mutant conditions (41). These observations strongly suggest that the critical role played by PLKs in MTOC maturation will be conserved from yeast to humans. From a mechanistic standpoint, the results obtained with cdc5-16 mutants suggest that the PBDs of mammalian PLKs will likely play critical roles in the control of MTOC functionality and in the maintenance of genome ploidy. In light of this, it is perhaps not surprising that PLK1 PBD mutations have been found in human tumor cell lines (16) and that many human cancers are characterized by misregulation of PLKs (4).

At the molecular level, the observation that not all Cdc5 substrates are affected to the same extent by the loss of PBD activity is highly interesting. Indeed, whereas some substrates are phosphorylated later or to a lesser extent in cdc5-16 cells (i.e., Bfa1, Brn1), others appear unaffected (i.e., Mcd1) or even hyper-phosphorylated Slk19). One likely reason for this phenotype is that loss of PBD activity will effectively increase the availability of Cdc5 for those substrates whose phosphorylation does not normally depend on the PBD activity. This increased availability of Cdc5 will lead to enhanced phosphorylation of “PBD-insensitive” substrates, whereas those substrates that normally require the PBD to be phosphorylated will be less so in cdc5 PBD mutants. This possibility is intriguing since it suggests that the phenotype associated with PBD inactivation may be the end result of two qualitatively different consequences: hyper-phosphorylation of some substrates and lack of phosphorylation of others. Thus, in addition to its role in stimulating substrate phosphorylation, another key function of the PBD may be to prevent over-phosphorylation of other substrates. This substrate over-phosphorylation could be mediated directly by Cdc5 or indirectly by another kinase. In any case, it would be advantageous to limit the phosphorylation of specific substrates when such modification affects the substrate activity in a quantitative manner. This putative inhibitory role for Cdc5 PBD is based on the assumption that Cdc5 kinase activity is limiting in cells. Whether this assumption and the PBD inhibition model are taking place in vivo remains to be shown experimentally.

Another key observation from our work and from that of others is that the MTOC functions of PLKs likely require the localization of these kinases to SPBs and centrosomes (5). Paradoxically, this localization to SPBs is a potential reason why Cdc5's role in the regulation of SPB functions may have been overlooked until now in budding yeast. Indeed, Cdc5 association to SPBs serves a functionally separate role in the regulation of mitotic exit (34), providing one reason other than SPB regulation for its enrichment at this location. In this context, the use of the Cdc5-16 mutant (W517F-H538A-K540M) has been instrumental to differentiate the SPB phenotypes from the mitotic exit phenotypes of Cdc5. A similar mutant has also been useful to identify a novel role for Cdc5 in mitotic spindle elongation (27). Importantly, defects in spindle elongation and SPB regulation are functionally distinct since it is possible to observe the former without the later, for example, in cells defective in microtubule motor activity (e.g., 42, 43). In the course of our study, we noticed that another allele that is partly defective in PBD activity, cdc5-12 (H538A-K540M), had a much weaker phenotype than the cdc5-16 mutant. In particular, the progression of cdc5-12 mutants from a 1n to 2n genomic content was slow and only half the population of mutant cells reached a 2n state after extended growth periods. Compared to cdc5-16 mutants, the weak mitotic defects of the cdc5-12 mutant (and of the similar cdc5-HK mutant (27)) indicate that this allele is not completely null in its PBD activity. It is also noteworthy that several is alleles of CDC5 carry point mutations affecting the PBD. From a functional standpoint, these alleles are not PBD-specific, however, because they severely reduce Cdc5 protein levels in vivo. As a consequence, these alleles should be considered defective in all biochemical activities of Cdc5, not exclusively in PBD activity.

Our ability to distinguish between the PBD and kinase-related functions of Cdc5 was made possible by the creation of kinase-specific mutations. It is remarkable that mutations in the F-helix of several essential kinases create conditional alleles of these kinases. This observation indicates that the F-helix region of EPKs is a mutagenic hotspot to create useful mutants with conditional activity. The ability to quantitatively modulate the enzymatic activity of kinases using predictable mutations has multiple applications in biology. In particular, F-helix mutants could be useful to understand the specific roles of multifunctional kinases and, in addition, may allow the identification of specific thresholds of kinase activity required for particular functions in cells. These mutations may thus be regarded as temperature-controlled rheostats to modulate the enzymatic activity of specific kinase domains in vivo.

### Materials and Methods

Abbreviations List

Unless otherwise stated, the following abbreviations apply:

BI, budding index; EPK, eukaryotic protein kinase; PBD, polo-box domain; SPB, spindle pole body; ts, temperature-sensitive; wt, wild-type.

1. Yeast Strains and Growth Conditions

All yeast strains used in this study are derivatives of W303 and are described in FIG. 7. Standard procedures were used for yeast culture, sporulation and tetrad dissection (Guthrie and Fink, 1991). Mutations in CDC5 and other kinases were generated by low-fidelity PCR amplification and/or site-directed mutagenesis of DNA fragments containing the coding sequence of a given kinase fused to the HIS3MX6 selection cassette. Mutant alleles of all kinases were inserted at their endogenous loci in yeast and the presence of the expected mutation(s) was confirmed by sequencing the relevant loci. The initial screen to identify kinase mutations was based on both random and targeted mutations derived from previously identified alleles of essential kinases. The temperature-sensitive (ts) growth phenotype of the various mutants was determined at 22° C., 30° C., and 38° C. Briefly, 5-fold dilution series of mutant yeast strains (first spot corresponds to a culture at OD₆₀₀ of 0.3) were spotted on solid medium and grown in temperature-controlled incubators (Binder KB115) for 48-72 hrs before taking a picture of the yeast spots. All experiments involving ts alleles grown in liquid cultures were performed at 38° C.

2. Protein Purification

Wild-type Cdc5, kinase-dead (KD) Cdc5-K110M, and Cdc5-77 were expressed as 9×HIS and StreptagII fusion proteins under the control of the GAL1 promoter using the multi-copy plasmids p632, p647, and p587, respectively. Cdc5 was purified from whole cell lysates by affinity chromatography on nickel-chelate and Streptactin-conjugated resins, as described previously (St-Pierre et al., 2009). For antibody production, Cdc5 was expressed as a 6×HIS-tagged protein in bacteria and purified from inclusion bodies on nickel-chelate resin (Qiagen). Monoclonal antibodies against Cdc5 were generated by Medimabs (Montreal, Canada).

3. In Vitro Kinase Assays

The kinase activity of Cdc5 and its derivatives was tested as described (St-Pierre et al., 2009), with minor modifications. To evaluate the impact of temperature on kinase activity, 40 ng of wt Cdc5 or 140 ng of Cdc5-D263Q were incubated 30 minutes at various temperatures in kinase reaction buffer containing 5 μg of dephospho-casein, 25 mM Tris-HCl pH 7.5, 2 mM DTT, 10 mM MgCl₂, 100 μM ATP and 1 μCi ATPγ³²P, 0.5 mM EDTA, 5 nM microcystin, 25 μM bromolevamisole oxalate, 5 mM β-glycerophosphate 1 mM AEBSF, 10 μM pepstatin A, 10 μM E-64, 0.2 mM tungstate, and 0.1 mM Na₃VO₄. To compare the absolute kinase activities of Cdc5 and its derivatives, 105 ng of purified wt Cdc5, Cdc5-D263Q, and Cdc5-K110M (i.e., Cdc5-KD, a kinase-dead mutant) were incubated at 30° C. for 30 mins in kinase reaction buffer. In some experiments, kinase assays were carried out using Cdc5 that has been pre-phosphorylated in vitro with Cdk1/cyclin B (Millipore). Mutant and wt versions of Cdc5 were phosphorylated to similar extent by Cdk1/cyclin B, as evidenced by similar levels of ³²P incorporation on Cdc5 under all conditions. For all kinase assays, phosphorylated substrates (casein and Cdc5 itself) were detected after SDS-PAGE using FLA-5000 imaging system (Fujifilm Life Science).

4. Protein Phosphorylation in Whole Cell Extracts and Western-Blotting

The in vivo phosphorylation status of epitope-tagged proteins was monitored by western-blotting following electrophoresis of total cellular extracts in gels containing NextGel acrylamide (Amresco) (St-Pierre et al., 2009). Endogenous Cdc5 from whole cell extracts was detected on western blots using a mixture of mouse monoclonal antibodies 11H12 and 4F10 (Medimabs, Montreal, Canada) at 1/500 dilution each. For detection of Clb2 in cell lysates, the protein was fused to a 3×HA tag and its levels were determined by western-blotting using the anti-HA 12CA5 antibody (Roche). For detection of Cdc15 in cell lysates, wt and mutant proteins were fused to a 13×MYC tag and their levels were determined by western-blotting using the anti-MYC 9E10 antibody (GenTex).

5. Flow Cytometry

Flow cytometry was performed as described (D'Amours and Jackson, 2001), with minor modifications. Briefly, cells were fixed by resuspension in 70% ethanol and stored at 4° C. for 24 to 48 hours. Cell pellets were washed with Tris buffer (50 mM Tris-HCl pH 7.5), briefly sonicated, incubated for 2 hours at 42° C. with RNAse A (Sigma) in Tris buffer, and 30 min at 50° C. with Proteinase K (Sigma) in Tris buffer. Cells were resuspended in fresh Tris buffer and stored at 4° C. for 1 to 24 hours. Immediately before flow cytometry analysis, genomic DNA was stained with 1 μM Sytox Green (Molecular Probes). Genomic DNA quantification was performed on a FACSCanto cytometer using FACSDiva software (BD Biosciences). Raw data were analyzed with FlowJo software.

7. Fluorescence Imaging

Indirect-immunofluorescence imaging of yeast mitotic spindles and Cdc14 localization was performed with rat anti alpha-tubulin antibody YOL1/34 and a goat polyclonal anti Cdc14 antibody (Santa Cruz Biotechnology), as previously described (St-Pierre et al., 2009). For the analysis of spindle position relative to the mother-bud axis, cells were released from an alpha-factor arrest and samples were collected every 15 min for labelling with an anti alpha-tubulin antibody, as described above. The fraction of cells carrying misaligned spindles was determined at the time-point corresponding to anaphase onset, as previously described (Snead et al., 2007). Visualization of URA3::tetO arrays in cells expressing tetR-GFP was performed in fixed yeast strains synchronized in G1 with α-factor. Visualization of SPBs in cells expressing Spc42/Spc72-GFP was performed essentially as described for URA3:.tetO arrays, except that yeast were synchronized in metaphase with nocodazole and DAPI was included in the mounting medium. 3×GFP-Cdc5 was visualized in live cells suspended in sterile water. All images were acquired using a Leica DM5500B epifluorescence microscope equiped with an EM-CCD camera (Hamamatsu). Volocity Software (Improvision, UK) was used for image acquisition. In order to optimize visualization of subcellular features, linear adjustments of image brightness and contrast were applied to primary/raw micrographs using Photoshop image editing software (Adobe).

8. Protein Phosphorylation in Whole Cell Extracts and Western-Blotting

The in vivo phosphorylation status of epitope-tagged proteins was monitored by western-blotting following electrophoresis of total cellular extracts in gels containing NextGel acrylamide (Amresco) (3). Endogenous Cdc5 from whole cell extracts was detected on western blots using a mixture of mouse monoclonal antibodies 11H12 and 4F10 (Medimabs, Montreal, Canada) at 1/500 dilution each. For detection of Clb2 in cell lysates, the protein was fused to a 3×HA tag and its levels were determined by western-blotting using the anti-HA 12CA5 antibody (Roche). For detection of Cdc15 in cell lysates, wt and mutant proteins were fused to a 13×MYC tag and their levels were determined by western-blotting using the anti-MYC 9E10 antibody (GenTex).

9. Live-Cell Imaging of SPBs

Preparation of yeast for time-lapse imaging was done as described previously (7). Images were acquired on a DeltaVision microscope using softWoRx software (Applied Precision) equipped with a planApo 1.4NA 100× Olympus objective and a CoolSnap HQ2 camera (Photometrics) at 1×1 binning. Yeast were time-lapsed for 45 minutes at 15 or 35 second intervals with 7 or 5 z-steps of 0.5 or 0.75 μm. Non-deconvolved maximum intensity projection of z-stacks was used for further analysis.

10. Image Data Analysis

For the quantification of Spc42-GFP fluorescence intensity at SPB, spots were manually identified and the integrated intensity was calculated using either ImageJ or Metamorph software. The image background was subtracted from the fluorescence intensity. For the analysis of the distribution of Spc42-GFP fluorescence intensity, spots of cycling cells were analysed on the first frame of each movie, so that photobleaching did not affect the measurements. Fluorescence intensity of control and mutant cells was normalized to the mean intensity of the control cells for each experiment. Therefore the mean intensity of the control cells was set to 1. The ratio of duplicated Spc42-GFP spots was calculated on one frame shortly (1-4 minutes) after duplication of the spot. For each cell, the ratio was obtained by dividing the highest signal by the smallest signal. Data were analysed using Excel.

### REFERENCES

1. Ubersax J A & Ferrell J E, Jr. (2007) Mechanisms of specificity in protein phosphorylation. Nat Rev Mol Cell Biol 8(7):530-541.
2. Mahajan A, et al. (2008) Structure and function of the phosphothreonine-specific FHA domain. Sci Signal 1(51):re12.
3. Pawson T & Kofler M (2009) Kinome signaling through regulated protein-protein interactions in normal and cancer cells. Curr Opin Cell Biol 21(2):147-153.
4. Archambault V & Glover D M (2009) Polo-like kinases: conservation and divergence in their functions and regulation. Nat Rev Mol Cell Biol 10(4):265-275.
5. Park J E, et al. (2010) Polo-box domain: a versatile mediator of polo-like kinase function. Cell Mol Life Sci 67(12):1957-1970.
6. Song S, Grenfell T Z, Garfield S, Erikson R L, & Lee K S (2000) Essential function of the polo box of Cdc5 in subcellular localization and induction of cytokinetic structures. Mol Cell Biol 20(1):286-298.
7. Seong Y S, et al. (2002) A spindle checkpoint arrest and a cytokinesis failure by the dominant-negative polo-box domain of Plk1 in U-2 OS cells. J Biol Chem 277(35):32282-32293.
8. Jang Y J, Lin C Y, Ma S, & Erikson R L (2002) Functional studies on the role of the C-terminal domain of mammalian polo-like kinase. Proc Natl Acad Sci U S A 99(4):1984-1989.
9. Elia A E, et al. (2003) The molecular basis for phosphodependent substrate targeting and regulation of Plks by the Polo-box domain. Cell 115(1):83-95.
10. Kannan N & Neuwald A F (2005) Did protein kinase regulatory mechanisms evolve through elaboration of a simple structural component? J Mol Biol 351(5):956-972.
11. Kornev A P, Taylor S S, & Ten Eyck L F (2008) A helix scaffold for the assembly of active protein kinases. Proc Natl Acad Sci USA 105(38):14377-14382.
12. Schutz A R & Winey M (1998) New alleles of the yeast MPS1 gene reveal multiple requirements in spindle pole body duplication. Mol Biol Cell 9(4):759-774.
13. Biggins S, et al. (1999) The conserved protein kinase Ipl1 regulates microtubule binding to kinetochores in budding yeast. Genes & Development 13(5):532-544.
14. Simanis V & Nurse P (1986) The cell cycle control gene cdc2+ of fission yeast encodes a protein kinase potentially regulated by phosphorylation. Cell 45(2):261-268.
15. Hermosilla G H, Tapia J C, & Allende J E (2005) Minimal CK2 activity required for yeast growth. Mol Cell Biochem 274(1-2):39-46.
16. Simizu S & Osada H (2000) Mutations in the Plk gene lead to instability of Plk protein in human tumour cell lines. Nat Cell Biol 2(11):852-854.
17. Mortensen E M, Haas W, Gygi M, Gygi S P, & Kellogg D R (2005) Cdc28-dependent regulation of the Cdc5/Polo kinase. Curr Biol 15(22):2033-2037.
18. Gardner R G, Nelson Z W, & Gottschling D E (2005) Degradation-mediated protein quality control in the nucleus. Cell 120(6):803-815.
19. Surana U, et al. (1993) Destruction of the CDC28/CLB mitotic kinase is not required for the metaphase to anaphase transition in budding yeast. EMBO Journal 12(5):1969-1978.
20. D'Amours D & Amon A (2004) At the interface between signaling and executing anaphase—Cdc14 and the FEAR network. Genes & Development 18(21):2581-2595.
21. Stegmeier F, Visintin R, & Amon A (2002) Separase, polo kinase, the kinetochore protein Slk19, and Spo12 function in a network that controls Cdc14 localization during early anaphase. Cell 108(2):207-220.
22. Snead J L, et al. (2007) A coupled chemical-genetic and bioinformatic approach to Polo-like kinase pathway exploration. Chem Biol 14(11):1261-1272.
23. Lee K S & Erikson R L (1997) Plk is a functional homolog of Saccharomyces cerevisiae Cdc5, and elevated Plk activity induces multiple septation structures. Mol Cell Bio! 17(6):3408-3417.
24. Shirayama M, Zachariae W, Ciosk R, & Nasmyth K (1998) The Polo-like kinase Cdc5p and the WD-repeat protein Cdc20p/fizzy are regulators and substrates of the anaphase promoting complex in Saccharomyces cerevisiae. EMBO Journal 17(5):1336-1349.
25. Garcia-Alvarez B, de Carcer G, Ibanez S, Bragado-Nilsson E, & Montoya G (2007) Molecular and structural basis of polo-like kinase 1 substrate recognition: Implications in centrosomal localization. Proc Nail Acad Sci U S A 104(9):3107-3112
26. Yoshida S, et al. (2006) Polo-like kinase Cdc5 controls the local activation of Rho1 to promote cytokinesis. Science 313(5783):108-111.
27. Chen Y C & Weinreich M (2010) Dbf4 regulates the Cdc5 Polo-like kinase through a distinct non-canonical binding interaction. J Biol Chem 285(53):41244-41254.
28. Knop M & Schiebel E (1997) Spc98p and Spc97p of the yeast gamma-tubulin complex mediate binding to the spindle pole body via their interaction with Spc110p. Embo J 16(23):6985-6995.
29. Sezen B, Seedorf M, & Schiebel E (2009) The SESA network links duplbation of the yeast centrosome with the protein translation machinery. Genes Dev 23(13):1559-1570.
30. Storchova Z, et al. (2006) Genome-wide genetic analysis of polyploidy in yeast. Nature 443(7111):541-547.
31. Park C J, et al. (2004) Requirement for Bbp1p in the proper mitotic functions of Cdc5p in Saccharomyces cerevisiae. Mol Biol Cell 15(4):1711-1723.
32. Hu F, et al. (2001) Regulation of the Bub2/Bfa1 GAP complex by Cdc5 and cell cycle checkpoints. Cell 107(5):655-665.
33. Park C J, et al. (2008) Requirement for the budding yeast polo kinase Cdc5 in proper microtubule growth and dynamics. Eukaryot Cell 7(3):444-453.
34. Park J E, et al. (2004) Novel functional dissection of the localization-specific roles of budding yeast polo kinase Cdc5p. Mol Cell Biol 24(22):9873-9886.
35. Hoepfner D, Schaerer F, Brachat A, Wach A, & Philippsen P (2002) Reorientation of mispositioned spindles in short astral microtubule mutant spc72Delta is dependent on spindle pole body outer plaque and Kar3 motor protein. Mol Biol Cell 13(4):1366-1380.
36. Thomas J H & Botstein D (1986) A gene required for the separation of chromosomes on the spindle apparatus in yeast. Cell 44(1):65-76.
37. Chial H J, Giddings T H, Jr., Siewert E A, Hoyt M A, & Winey M (1999) Altered dosage of the Saccharomyces cerevisiae spindle pole body duplication gene, NDC1, leads to aneuploidy and polyploidy. Proc Natl Acad Sci U S A 96(18):10200-10205.
38. Hoepfner D, Brachat A, & Philippsen P (2000) Time-lapse video microscopy analysis reveals astral microtubule detachment in the yeast spindle pole mutant cnm67. Mol Biol Cell 11(4):1197-1211.
39. Glover D M (2005) Polo kinase and progression through M phase in Drosophila: a perspective from the spindle poles. Oncogene 24(2):230-237.
40. Sunkel C E & Glover D M (1988) polo, a mitotic mutant of Drosophila displaying abnormal spindle poles. J Cell Sci 89 (Pt 1):25-38.
41. Oshimori N, Ohsugi M, & Yamamoto T (2006) The Plk1 target Kizuna stabilizes mitotic centrosomes to ensure spindle bipolarity. Nat Cell Biol 8(10):1095-1101.
42. Straight A F, Sedat J W, & Murray A W (1998) Time-lapse microscopy reveals unique roles for kinesins during anaphase in budding yeast. Journal of Cell Biology 143(3):687-694.
43. Tytell J D & Sorger P K (2006) Analysis of kinesin motor function at budding yeast kinetochores. J Cell Biol 172(6):861-874.
44. St-Pierre J, et al. (2009) Polo kinase regulates mitotic chromosome condensation by hyperactivation of condensin DNA supercoiling activity. Mol Cell 34(4):416-426.
45. Maddox P S, Bloom K S, & Salmon E D (2000) The polarity and dynamics of microtubule assembly in the budding yeast Saccharomyces cerevisiae. Nat Cell Biol 2(1):36-41.
46. Sullivan M, Lehane C, & Uhlmann F (2001) Orchestrating anaphase and mitotic exit: separase cleavage and localization of Slk19.[see comment]. Nature Cell Biology 3(9):771-777.
47. Michaelis C, Ciosk R, & Nasmyth K (1997) Cohesins: chromosomal proteins that prevent premature separation of sister chromatids. Cell 91(1):35-45.

Although the above description relates to a specific preferred embodiment as presently contemplated by the inventor, it will be understood that the invention in its broad aspect includes functional equivalents in a variety of species of the elements described herein.

1. A temperature-sensitive eukaryotic protein kinase polypeptide comprising at least one mutated amino acid residue located in its subdomain IX.

2. The polypeptide according to claim 1, wherein the at least one mutated residue is the conserved aspartate and/or the residue immediately C-terminal to the conserved glycine residue in the subdomain IX.

3. (canceled)

4. The polypeptide according to claim 2, wherein the conserved aspartate residue is mutated to an asparaqine or a glutamine residue.

5. The polypeptide according to claim 2, wherein the residue immediately C-terminal to the conserved glycine residue is mutated to an aspartic acid or a qlutamic acid residue.

6. (canceled)

7. The polypeptide according to claim 1, wherein the subdomain IX comprises one or the following amino acid sequences SEQ ID NO: 1; SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; or SEQ ID NO: 12.

8-9. (canceled)

10. The polypeptide according to claim 1, wherein the kinase is of the following family of kinases: AMPK, Aurora kinases, BUB1, CAK1, Cyclin-dependent kinases (CDKs) Calmodulin kinase, Casein kinases 1 and 2, CHK1, CHK2, MAP kinases, MAP kinase kinase, MPS1, NDR, or p21-activated kinase.

11. A method of producing a mutated eukaryotic protein kinase, the method comprising:
a) transfecting a cell with a nucleic acid encoding the polypeptide according to claim 1; and
b) culturing the transfected cells under conditions for expressing the polypeptide.

12. A nucleic acid encoding a temperature-sensitive eukaryotic protein kinase polypeptide comprising at least one mutated amino acid residue located in its subdomain IX.

13. The nucleic acid according to claim 12, wherein the at least one mutated residue is the conserved aspartate and/or the residue immediately C-terminal to the conserved glvcine residue in the subdomain IX.

14. The nucleic acid according to claim 12, wherein the eukaryotic protein kinase is from a non-mammalian eucaryote.

15. The nucleic acid according to claim 14, whereinin the non-mammalian eucarvote is a yeast strain.

16. The nucleic acid according to claim 12, wherein the eukaryotic protein kinase is from a mammalian eucaryote.

17. A vector comprising the nucleic acid according to claim 12.

18. A cell comprising the nucleic acid according to claim 12.

19. The cell according to claim 18, wherein said cell is an eukaryotic cell.

20-28. (canceled)

29. The polypeptide according to claim 4, wherein the conserved aspartate residue is mutated to a glutamine residue.

30. The polypeptide according to claim 5, wherein the residue immediately C-terminal to the conserved glycine residue is mutated to a glutamic acid residue.

31. A method of identifying a temperature-sensitive mutant of an eukaryotic protein kinase polypeptide, said method comprising:
a) introducing at least one mutation in a nucleic acid encoding the eukaryotic protein kinase, wherein said at least one mutation leads to an amino acid change in subdomain IX of the eukaryotic protein kinase polypeptide;
b) culturing a cell transfected with the mutated nucleic acid of a) at different temperatures; and
c) testing the activity of the mutated eukaryotic protein kinase polypeptide at the different temperatures to identify a temperature-sensitive mutant of an eukaryotic protein kinase polypeptide.

32. The nucleic acid according to claim 13, wherein the conserved aspartate residue is mutated to an asparagine or a glutamine residue.

33. The nucleic acid according to claim 13, wherein the residue immediately C-terminal to the conserved glycine residue is mutated to an aspartic acid or a glutamic acid residue.
